(19) United States
(12) Patent Application Publication
(10) Pub. No.: US 2006/0039888 A1
(43) Pub. Date: Feb. 23, 2006

(54) Deodorizing method and apparatus, and a vacuum cleaner using same

(75) Inventors: Seiji Yamaguti, Konan-shi (JP); Masakazu Onda, Higashiomi-shi (JP)

Correspondence Address: BACON & THOMAS, PLLC, 625 SLATERS LANE, FOURTH FLOOR, ALEXANDRIA, VA, 22314, US

(73) Assignee: Matsushita Electric Industrial Co., Ltd., Osaka (JP)

(21) Appl. No.: 11/192,068

(22) Filed: Jul. 29, 2005

(30) Foreign Application Priority Data
Aug. 18, 2004 (JP) 2004-238110

(51) Int. Cl. A61L 9/01

(52) U.S. Cl. 424/76.1

(57) ABSTRACT

An apparatus for deodorizing dust particles includes a housing chamber, a powdering/granulating unit installed in the housing chamber, a deodorant disposed to be movable relative to the powdering/granulating unit, and a driving unit for driving the powdering/granulating unit or the deodorant. The deodorant is formed into granules or powders by a relative movement of the deodorant and the powdering/granulating unit, and thus obtained deodorant in granule or powder form is mixed with the dust particles.

### FIELD OF THE INVENTION

The present invention relates to a method and apparatus for eliminating odor of dust particles, and a vacuum cleaner which employs the method and apparatus.

### BACKGROUND OF THE INVENTION

With regard to a conventional vacuum cleaner employing a deodorizing method and apparatus, dust particles sucked in by the vacuum cleaner are collected in, for example, a porous bag or dust trap filter provided in a dust chamber, and deodorization of the dust particles is done by deodorant impregnated in the porous bag or the dust trap filter (see, for example, Japanese Patent Laid-Open Application No. H5-70871).

Such a conventional deodorizing mechanism, however, cannot provide a sufficient deodorization effect because the contact surface area between the deodorant and the dust particles is insufficient and the contact period therebetween is also too short. As a result, depending on the type or accumulated amount of dust particles, the exhaust air from the vacuum cleaner sometimes has an offensive odor, which tends to be unpleasant for a user.

### SUMMARY OF THE INVENTION

It is, therefore, an object of the present invention to provide a high-efficiency deodorizing method and apparatus capable of increasing the contact surface area and contact period between dust particles and a deodorant by way of blending dust particles with granulated or powdered deodorant, and a vacuum cleaner using same.

In accordance with a first aspect of the present invention, there is provided a method for deodorizing dust particles by mixing the dust particles with a deodorant in the form of granules or powders. As a result, the contact surface area and the contact period between dust particles and deodorant can be increased considerably, thus greatly improving the deodorization efficiency.

In accordance with a second aspect of the present invention, there is provided a method for deodorizing dust particles including the steps of: forming a solid deodorant into granules or powders, and mixing the dust particles with the deodorant in the form of granules or powders. When a solid deodorant is used, the density of deodorant is greater than granular or powder type deodorant although they have the same volumes. Hence, using a solid deodorant provides a longer period of deodorization which is more convenient for the user.

In accordance with a third aspect of the present invention, there is provided an apparatus for deodorizing dust particles including a housing chamber, a powdering/granulating unit installed in the housing chamber, a deodorant disposed to be movable relative to the powdering/granulating unit, and a driving unit for driving the powdering/granulating unit or the deodorant, wherein the deodorant is formed into granules or powders by relative movements of the deodorant and the powdering/granulating unit, and thus such deodorant in granule or powder form is mixed with the dust particles. Therefore, when the above configuration is applied to an apparatus for requiring deodorization therefrom, the contact surface area and the contact period between dust particles and a deodorant can be increased considerably, thus greatly improving the deodorization efficiency as the first aspect.

In accordance with a fourth aspect of the present invention, there is provided an apparatus for deodorizing dust particles including a dust chamber for colleting dust particles therein, a deodorant to be formed into granules or powders, and a mixing unit for mixing the dust particles with the deodorant in the form of granules or powders. In the above deodorizing apparatus, the contact area and the contact period between dust particles and deodorant can be increased considerably, thus greatly improving the deodorization efficiency as the first aspect.

In accordance with a fifth aspect of the present invention, there is provided a vacuum cleaner including an electric blower for suctioning dust particles, a dust chamber for collecting the dust particles therein, and a deodorizing apparatus for supplying a deodorant in the form of granules or powders into the dust chamber or at upstream location of the dust chamber, wherein the deodorant in the form of granules or powders is mixed with the dust particles by a suctioning force of the electric blower. With the above configuration, the vacuum cleaner having excellent deodorization function is obtained. Simultaneously, since electric blower is used to blend odorant and dust particles, the separate mixing unit is not needed, simplifying the structure of the vacuum cleaner.

In accordance with a sixth aspect of the present invention, there is provided a vacuum cleaner including a housing chamber is installed in and made to communicate with the dust chamber or a dust particle passageway upstream of the dust chamber, wherein the deodorant is loaded in the housing chamber to supply the deodorant in the form of granules or powders into the dust chamber. In the above explained configuration, since odorant can be supplied in granule or power form to the dust particle passageway by suction force of the electric blower, simplifying the structure of the vacuum cleaner.

In accordance with a seventh aspect of the present invention, there is provided a vacuum cleaner including a powdering/granulating unit for forming a solid deodorant into granules or powders, wherein the solid deodorant is formed into granules or powders by the powdering/granulating unit. When a solid deodorant is used, the density of deodorant is higher compared to granular or powder deodorant even though they have the same volumes. Hence, the solid deodorant can performs deodorization for a long time, thus providing greater convenience to the user as the second aspect.

In accordance with an eighth aspect of the present invention, there is provided a vacuum cleaner wherein the deodorant and the powdering/granulating unit are disposed to be movable relative to each other so that at least one of the deodorant and the powdering/granulating unit is moved by drawing and moving action a main body of the vacuum cleaner, and the deodorant is formed into granules or powders by the movement of said at least one of the deodorant and the powdering/granulating unit. Therefore, deodorant can be formed in granule or power form only by drawing and moving the vacuum cleaner, simplifying the structure of the vacuum cleaner.

In accordance with a ninth aspect of the present invention, there is provided a vacuum cleaner, wherein the deodorant and the powdering/granulating unit are disposed to be movable relative to each other, and at least one of the deodorant and the powdering/granulating unit is driven manually so that the deodorant can be formed into granules or powders at any time a user whishes. With this configuration, since the deodorant can be anytime formed into granules or powders, deodorization can be performed at any time a user wants.

In accordance with a tenth aspect of the present invention, there is provided a vacuum cleaner, wherein the deodorant and the powdering/granulating unit are disposed to be movable relative to each other, and further comprising an electric driving unit for driving at least one of the deodorant and the powdering/granulating unit and a controller for controlling the electric driving unit to be operated constantly, periodically or intermittently to form the deodorant into granules or powders. Therefore, deodorization is simply carried out.

In accordance with an eleventh aspect of the present invention, there is provided a vacuum cleaner, wherein the deodorant and the powdering/granulating unit are disposed to be movable relative to each other, and further comprising a cord reel for driving at least one of the deodorant and the powdering/deodorizing unit so that the deodorant is formed into granules or powders when a cord is extracted or retracted. In this configuration, since the user can carry out deodorization simply by extracting or retracting the power cord, the deodorization function is implemented easily by the user.

In accordance with a twelfth aspect of the present invention, there is provided a vacuum cleaner, wherein the housing chamber is installed in the dust chamber or at a location above the dust particle passageway upstream of the dust chamber. In this configuration, deodorant in powder or granule form is supplied to the dust particle passageway by gravity, simplifying the structure of the vacuum cleaner. In this configuration, deodorant in powder or granule form is supplied to the dust particle passageway by gravity, simplifying the structure of the vacuum cleaner.

In accordance with a thirteenth aspect of the present invention, there is provided a vacuum cleaner, wherein the housing chamber is provided with an opening for replacing the deodorant with a new one. Therefore, deodorant can be replenished appropriately, thus providing greater convenience to the user.

In accordance with a fourteenth aspect of the present invention, there is provided a vacuum cleaner including a deodorant amount indicator for indicating a residual amount of the deodorant. With this configuration, timing of replenishing deodorant can be recognized with easy, thus providing greater convenience to the user.

In accordance with a fifteenth aspect of the present invention, there is provided the vacuum cleaner including an antibacterial agent and/or an aromatic compound as well as the deodorant, wherein the deodorant, the antibacterial agent and/or the aromatic compound is formed into granules or powders to be mixed with the dust particles. With this configuration, sterilization and deodorization are provided, thus providing a highly hygienic environment.

### BRIEF DESCRIPTION OF THE DRAWINGS

The above and other objects and features of the present invention will become apparent from the following description of preferred embodiments given in conjunction with the accompanying drawings, in which:

FIG. 1 is a schematic configuration view of a vacuum cleaner in accordance with a first preferred embodiment of the present invention;

FIG. 2 presents a cross sectional view of the vacuum cleaner of FIG. 1;

FIG. 3 sets forth an enlarged perspective view of the major components of the vacuum cleaner of FIG. 1;

FIG. 4 provides a cross sectional view of the major components of a vacuum cleaner in accordance with a second preferred embodiment of the present invention;

FIG. 5 shows a cross sectional view of a vacuum cleaner in accordance with a third preferred embodiment of the present invention;

FIG. 6 is a perspective view of the vacuum cleaner in accordance with a fourth preferred embodiment of the present invention;

FIG. 7 presents a cross sectional view of a main body of the vacuum cleaner of FIG. 6;

FIG. 8 sets forth a cross sectional view of a cord reel of the vacuum cleaner of FIG. 6;

FIG. 9 provides a perspective view of the main body of the vacuum cleaner of FIG. 6;

FIG. 10 shows a view for setting forth an internal power transmission of the vacuum cleaner of FIG. 6;

FIG. 11 is an exploded perspective view of a deodorant receiving unit in accordance with a fourth preferred embodiment of the present invention; and

FIG. 12 presents a cross sectional view of the deodorant accommodating portion in accordance with a fourth preferred embodiment of the present invention.

### DETAILED DESCRIPTION OF THE PREFERRED EMBODIMENTS

Hereinafter, preferred embodiments of the present invention will be described with reference to the accompanying drawings. Here, it is to be noted that the present invention is not limited thereto.

### First Preferred Embodiment

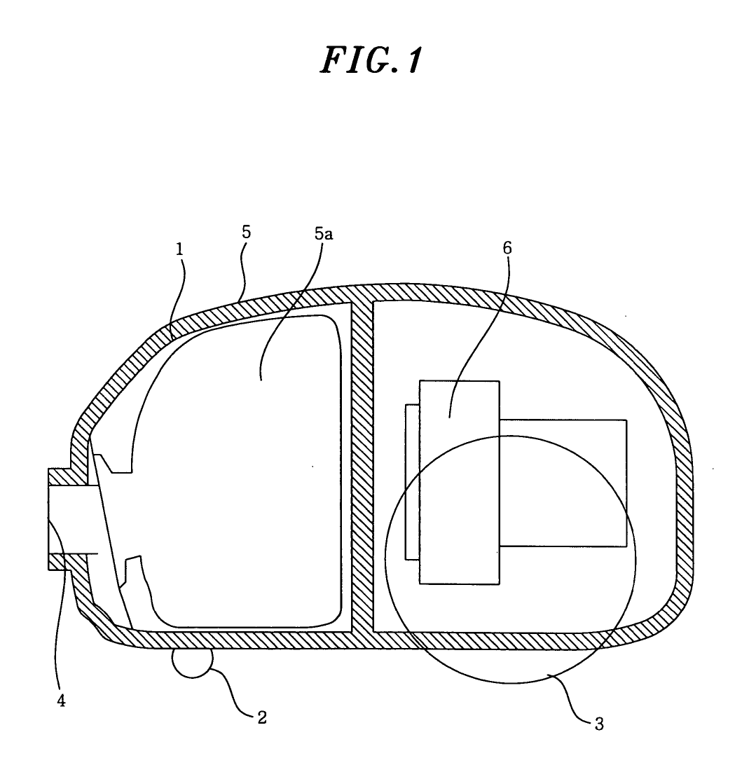

FIG. 1 illustrates a configuration of a common electric vacuum cleaner. As shown in the figure, front swivel caster 2 and a pair of rear wheels 3 are rotatably supported at main body 1 of the vacuum cleaner. Provided at a front portion of main body 1 is suction air inlet 4 for suctioning dirt carrying air, and a suction nozzle (not shown) for sucking dust particles is connected to suction air inlet 4 via an extension tube (not shown) and a hose (not shown). Moreover, main body 1 includes dust chamber 5 and electric blower 6 installed downstream of suction air inlet 4 in that order.

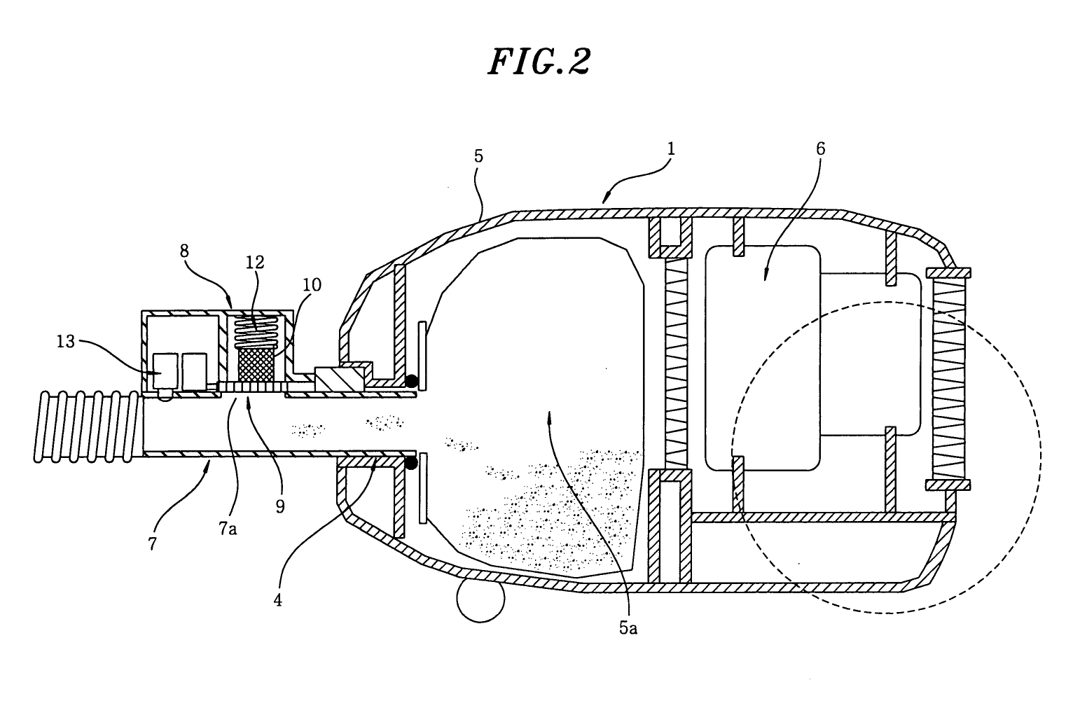

Referring to FIG. 2, dust chamber 5 has dust bag 5a for collecting dust particles therein and electric blower 6 generates suction air stream for sucking up dust particles. Suction air inlet 4 communicates with dust chamber 5 and is connected to suction tube 7 (to be connected to a hose (not shown) as one body) for sucking and moving dust particles into main body 1 from floor or the like. Vacuum cleaning is carried out as follows. Electric blower 6 operates to create suction, a partial vacuum inside the vacuum cleaner. The ambient air carrying dust particles pushes itself into the vacuum cleaner through suction tube 7. As a result, the dust particles are collected and trapped in dust bag 5a provided in dust chamber 5. The dirt-filled air subsequently free of dust particles after going through a dirt trap unit is then exhausted from main body 1 externally.

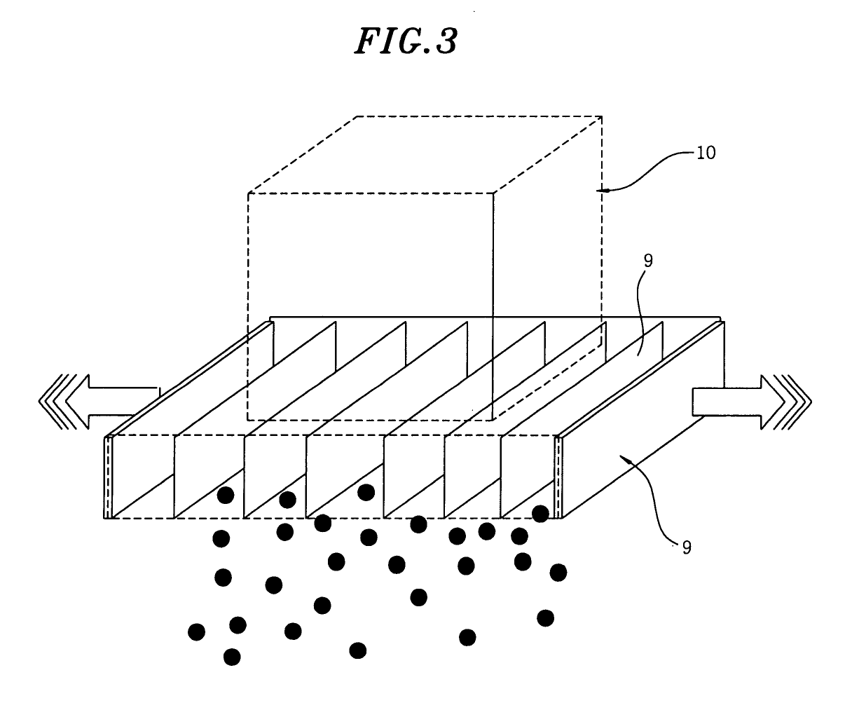

Next a deodorizing method and apparatus employed in a vacuum cleaner with the above configuration will be described in accordance with a first preferred embodiment of the present invention. As shown in FIG. 2, installed above suction tube 7 is housing chamber 8 communicating therewith via communication opening 7a. Disposed within housing chamber 8 above communication opening 7a is powdering/granulating unit 9 as illustrated in FIG. 3, which is formed of a plurality of thin metal plates arranged at equal intervals and held in place by a frame-shaped jig. Deodorant 10, for example, solid zeolite is mounted on top of powdering/granulating unit 9. Deodorant 10 and powdering/granulating unit 9 are movable relative to each other. In the first preferred embodiment, deodorant 10 is configured to be freely movable both horizontally and vertically.

Moreover, reference numeral 13 represents a dirt detection unit disposed upstream of communication opening 7a. Dirt detection unit 13 is formed of, e.g., an infrared sensor.

The operation of the deodorizing mechanism with the above configuration is as follows. In accordance with the first preferred embodiment, when a user moves main body 1 of the vacuum cleaner for cleaning, deodorant 10 is allowed to shake back and forth or up and down on thin metal plates 9a of powdering/granulating unit 9, in response to the horizontal or vertical movement of main body 1. As a result, the bottom surface of deodorant 10 contacting thin metal plates 9a is shaved off by a certain width, and deodorant solid is powdered or granulated because the bonds between binder and zeolite of the cut portion of deodorant 10 become broken due to the impact against thin metal plates 9a as deodorant 10 is cut off. Deodorant 10 in powder or granule form then falls down into suction tube 7 by gravity. At this time, since housing chamber 9 communicates with suction tube 7 via communication opening 7a, powdered or granulated deodorant 10 gets sucked into suction tube 7 powerfully by the suctioning force of electric blower 6 for suctioning dust particles.

Then, powdered or granulated deodorant 10 sucked into suction tube 7 is mixed with dust particles by the suction air stream generated due to the suctioning force of electric blower 6, finally reaching dust bag 5a in dust chamber 5. Powdered or granulated deodorant 10 is then further mixed with dust particles accumulated in dust chamber 5. At this time, if observed microscopically, each of the dust particles is surrounded by granules or powders of deodorant 10, so that the contact surface area between each dust particle and deodorant is greatly increased. Such arrangement of deodorant 10 and the dust particles is maintained in dust bag 5a. Consequently, a deodorization effect of deodorant 10 becomes maximized, effectively eliminating offensive odor. For example, even when pet hairs with offensive odor were sucked into the vacuum cleaner, the exhaust air from main body 1 had very little smell.

Moreover, since it is possible to powder or granulate the deodorant simply by way of drawing and moving main body 1 of the vacuum cleaner, an additional structure for forming the deodorant into powder or granules is not necessary, thus allowing a simple structure of the vacuum cleaner. Furthermore, since the deodorant and the dust particles are mixed by the suction air stream generated by electric blower 6, which is a basic component of a vacuum cleaner, an additional structure for mixing the deodorant and the dust particles is not needed, thereby further contributing to the structural simplification of the vacuum cleaner.

As described above, in the deodorizing mechanism in accordance with the first preferred embodiment of the present invention, by forming a solid deodorant into powders or granules and mixing dust particles with thus powdered or granulated deodorant, a highly improved deodorization effect which can not be obtained by the conventional deodorizing mechanism can be achieved. Furthermore, by applying the deodorizing mechanism to vacuum cleaners, unpleasant smell of exhaust air from the vacuum cleaner can be eliminated, thus making the experience of using the vacuum more pleasant for a user.

Although the first preferred embodiment of the invention has been described with respect to powdering/granulating unit 9 installed at the upstream of dust chamber 5, it is to be understood that it can be disposed in dust chamber 5.

### Second Preferred Embodiment

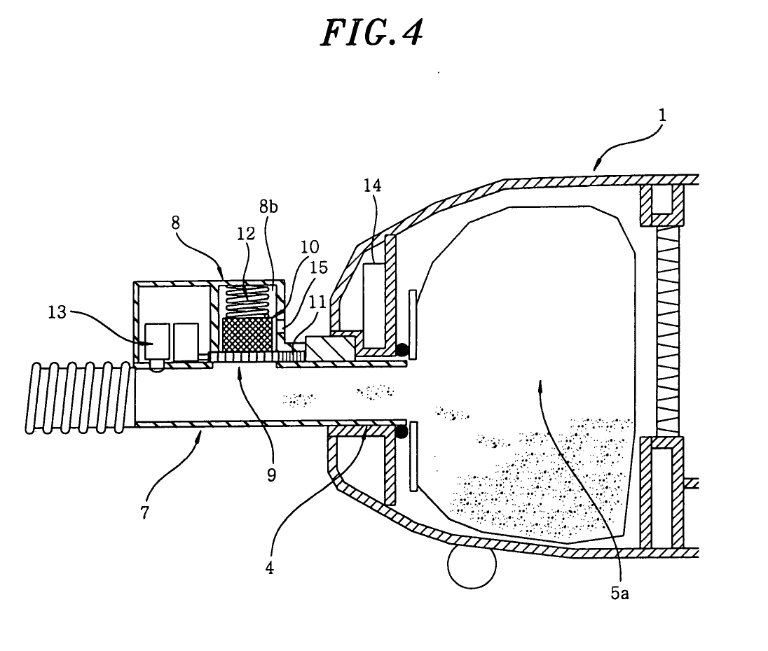

FIG. 4 illustrates a second preferred embodiment of the present invention. Here, explanation of parts identical to those of the first preferred embodiment will be omitted; and instead distinctive parts will be focused and elaborated. The technical feature of the second preferred embodiment resides in that powdering/granulating unit 9 for powdering or granulating deodorant 10 is driven. In FIG. 4, driving unit 11 such as vibrator drives powdering/granulating unit 9 under the control of controller 14 provided at an appropriate location in main body 1 of the vacuum cleaner. Controller 14 is energized by plugging in main body 1 or turning on an operation switch (not shown) of main body 1. Moreover, elastic member 12 such as a spring pushes deodorant 10 toward powdering/granulating unit 9 such that deodorant 10 is pressed against powdering/granulating unit 9.

In the above configuration, when main body 1 is energized or the operation switch is turned on, controller 14 allows driving unit 11 to vibrate powdering/granulating unit 9 back and forth according to a preset sequence, whereby deodorant 10 is powdered or granulated and is supplied into suction tube 7 to be mixed with dust particles, thus deodorizing them as in the first preferred embodiment.

In the above configuration, controller 14 is configured to drive powdering/granulating unit 9 continuously throughout a cleaning operation or periodically or intermittently during the vacuum cleaning. Moreover, even though the vacuum cleaner is not vacuuming, controller 14 is set to drive powdering/granulating unit 9 periodically or intermittently if the vacuum cleaner is plugged in. Therefore, the deodorization can be carried out as necessary to maximize its effect. Further, since the user can form the deodorant into granules or powders automatically just by, for example, turning on a deodorization switch (not shown), its application is more accommodating.

Moreover, by maintaining deodorant 10 in a pressurized contact with powdering/granulating unit 9 by means of elastic member 12, the amount of deodorant cut, that is, the amount of deodorant formed into granules or powders can be made steady. Also, the presence and the function of elastic member 12 prevents powdering/granulating unit 9 from shaking vertically, so that the operation of powdering/granulating unit 9 can be stabilized.

Furthermore, in the second preferred embodiment, opening 8b that can be opened or closed by a lid (not shown) is provided at an upper portion of housing chamber 8 to replenish deodorant 10. Thus, when deodorant 10 is all used up, it can be replenished through opening the lid.

In FIG. 4, there is further provided deodorant amount indicator 15 for informing a user of the residual amount of deodorant 10. In FIG. 4, it is implemented by forming a part of the lid with a transparent material such that the inside of housing chamber 8 can be viewed from outside. Through the transparent portion of the lid, the residual amount of deodorant 10 can be observed, and its replenishing can be done appropriately. Alternatively, deodorant amount indicator 15 can be formed of a sensor for detecting the residual amount of deodorant 10 and a display unit such as a LED for displaying it.

Opening 8b and deodorant amount indicator 15 can be applied to the first preferred embodiment described above or to a third preferred embodiment to be explained below.

### Third Preferred Embodiment

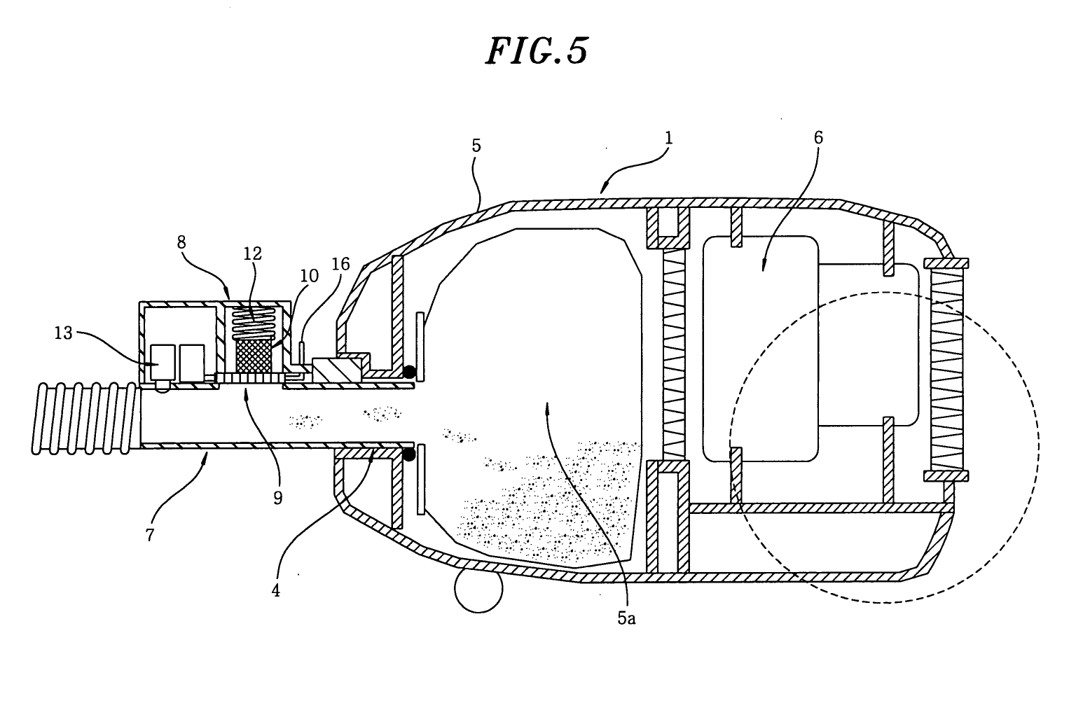

FIG. 5 describes a third preferred embodiment of the present invention. Explanation of parts identical to those of the first or the second preferred embodiment will be omitted, while focusing on and elaborating only distinctive parts. In accordance with the third preferred embodiment, powdering/granulating unit 9 is vibrated manually using manipulation unit 16.

When deodorization is necessary, a user can vibrate powdering/granulating unit 9 back and forth by using manipulation unit 16 to form deodorant 10 into granules or powders. With this configuration, the user can perform deodorization when desired.

Further, although another mechanism for vibrating powdering/granulating unit 9 is not shown in the figure, it can be implemented by connecting powdering/granulating unit 9 to a cord reel of the power cord such that powdering/granulating unit 9 is vibrated by the action of extracting or retracting the power cord. At this time, since the user can carry out deodorization simply by extracting or retracting the power cord, the deodorization function is implemented easily by the user.

In the above-described preferred embodiments of the present invention, an adsorbent is used as deodorant 10. For example, a preferred deodorant includes active carbon capable of adsorbing various odors caused by dust particles including low grade fatty acid, ammonia, aldehyde, or so forth. Also, a zeolite-based adsorbent having the same effect can be used as adsorbent, and in particular hydrophobic zeolite is preferable.

Further, it is also possible to add an antibacterial agent or an aromatic compound to deodorant 10. Antibacterial components of the antibacterial agent preferably include silver-based, zinc-based or copper-based components for inorganic substances; and allyl isothiocyanate or catechin for organic substances, as these compounds are highly effective antibiotics against bacteria and fungi. The silver-based material is particularly preferred because it can provide a great antibacterial effect with only a small amount, while providing sterilization as well as deodorization, thus providing a highly hygienic environment.

Furthermore, if powders of deodorant such as zeolite or active carbon are contained in dust bag 5a, a dust trap filter downstream of dust bag 5a or a wall serving as a flow path of an exhaust air stream, the overall deodorization efficiency can be further improved without increasing pressure loss.

In accordance with the present invention descried above, the contact surface area and the contact period between dust particles and deodorant can be increased considerably, thus achieving great improvement in deodorization efficiency. Therefore, the present invention has many advantages when it is applied to vacuum cleaners, food waste treating systems, and so forth.

### Fourth Preferred Embodiment

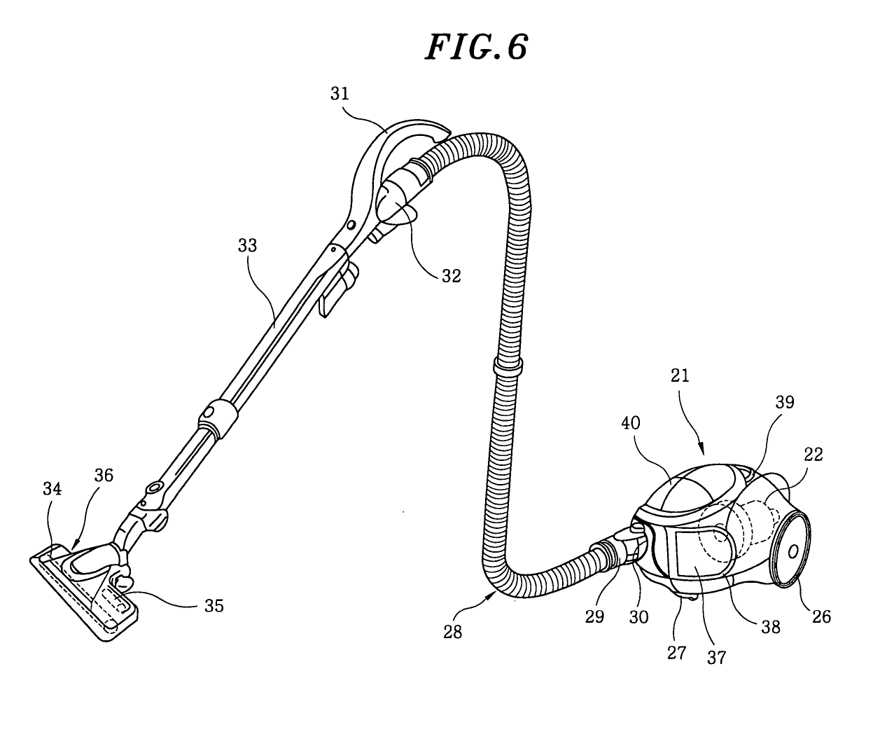

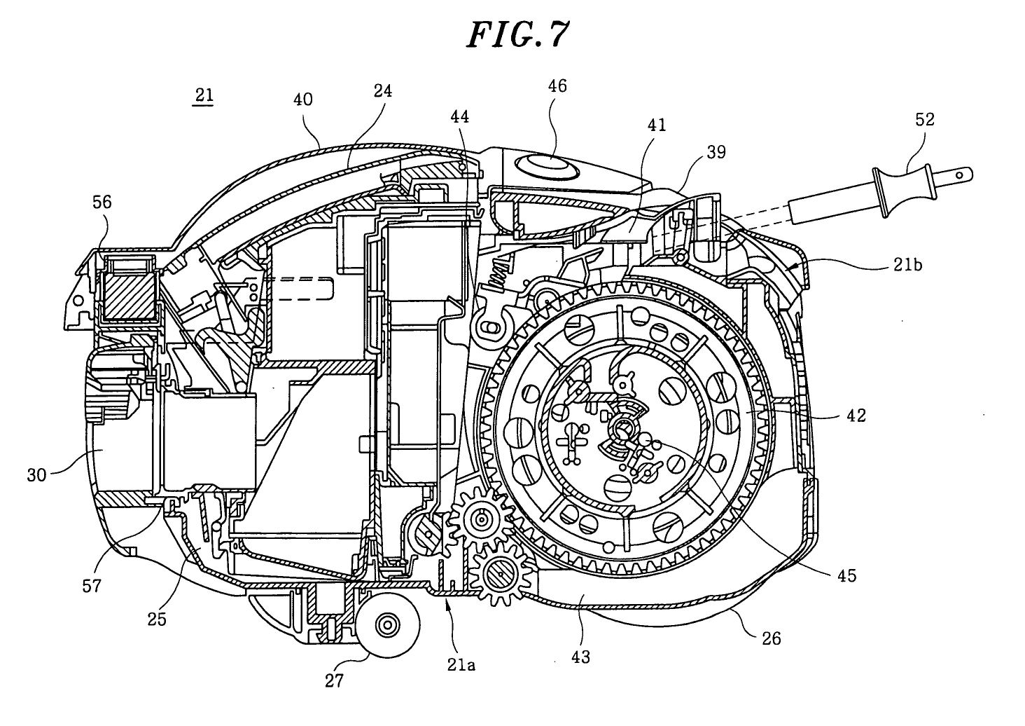

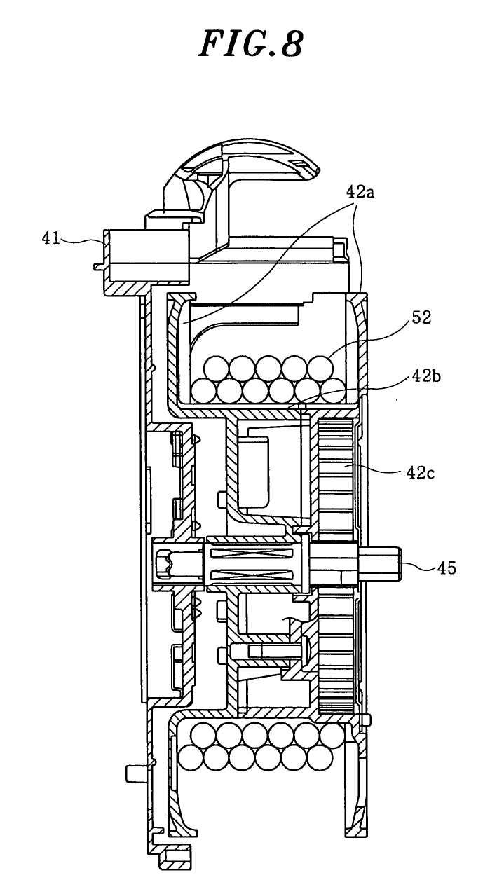

A vacuum cleaner in accordance with fourth preferred embodiment of the present invention will now be described with reference to FIG. 6 to FIG. 12.

As shown in FIGS. 6 to 9, reference numeral 21 represents a main body of a vacuum cleaner. Provided in the rear portion of main body 21 is electric blower chamber 23 mounting therein electric blower 22 and detachably provided in the front portion thereof is dust box receiving portion 25 for accommodating dust box unit 24 for separating and collecting dust particles. Further, two wheels 26 are rotatably installed on both lower side surfaces of a rear portion of main body 21 and swivel caster 27 is installed at a front bottom surface thereof. In addition, provided at a front portion of main body 21 is suction air inlet to which one end of connection pipe 29 is detachably connected. The other end of connection pipe 29 is connection to one end of hose 28.

Installed at the other end of hose 28 is leading pipe 32 with handle 31 which is gripped by user when using the vacuum cleaner. Reference numeral 33 represents extension tube functioning as a flexible member or a joint member. Extension tube 33 has one end detachably connected to leading pipe 32 on the downstream side and the other end detachably connected to suction head 36. Suction head 36 includes rotation brush 34 for lifting up dust particles and motor 35 for rotating rotation brush 34.

Reference numeral 37 represents transparent windows which are respectively provided at both sides of main body 21. Through transparent windows 37, transparent outer sides of dust box unit 24 included in dust box receiving portion 25 can be viewed from outside. Reference numeral 38 represents a bumper for preventing furniture or house walls from getting damaged when main body 21 collides therewith. Bumper 38 is made of resilient material and extends from a front portion of main body 21 to both sides thereof. Further, each of windows 37 is surrounded by resilient material.

Reference numeral 39 represents a main body handle for carrying main body 21. Main body handle 39 is raised when carrying main body 21. Further, main body handle 39 is rotatably installed at top of main body 21 so that its rotational center is located on a line passing through the center of main body.

Reference numeral 40 represents lid for accommodating dust box unit 24, lid 40 being rotatably, axially supported at main body 21.

The outer wall of main body 21 includes lower body 21a forming a lower part thereof and upper body 21b for covering lower body 21a. Lower body 21a has at rear portion of main body 21 cord reel receiving portion 43 for incorporating cord reel (power cord winding device) 42 and electric blower chamber 23. Reference numeral 41 represents a partition wall for separating electric blower chamber 23 and cord reel receiving portion 43 wherein cord reel 42 is rotatably, axially supported at partition wall 41 through axle supporting portion 45.

Cord reel 42 for winding power cord 52 includes drum portion 42b having at its upper and lower part flanges 42a and resilient member 42c for exerting pressing force against drum portion 42b in a direction that winds power cord 52.

Reference numeral 46 represents a rewind button for rewinding extracted power cord 52 onto cord reel 42 against the pressing force of resilient members 42c incorporated in cord reel 42. By pressing rewind button 46, brake roller 44 which prevents cord reel 42 from being rotated is released from cord reel 42 which in turn allows cord reel 42 to rewind power cord 52 by means of the pressing force of resilient members 42c.

Moreover, provided above suction air inlet 30 is deodorant accommodating portion 56.

The front portion of the electric blower 22 is supported by front support (not shown) and the rear portion thereof is supported by rear support (not shown) in electric blower chamber 23. Reference numeral 49 represents a partition wall for separating electric blower chamber 23 and dust box receiving portion 25. Approximately central portion of partition wall 49 is provided with lattice-shaped communication holes 50 communicating with air suction opening 22a of electric blower 22.

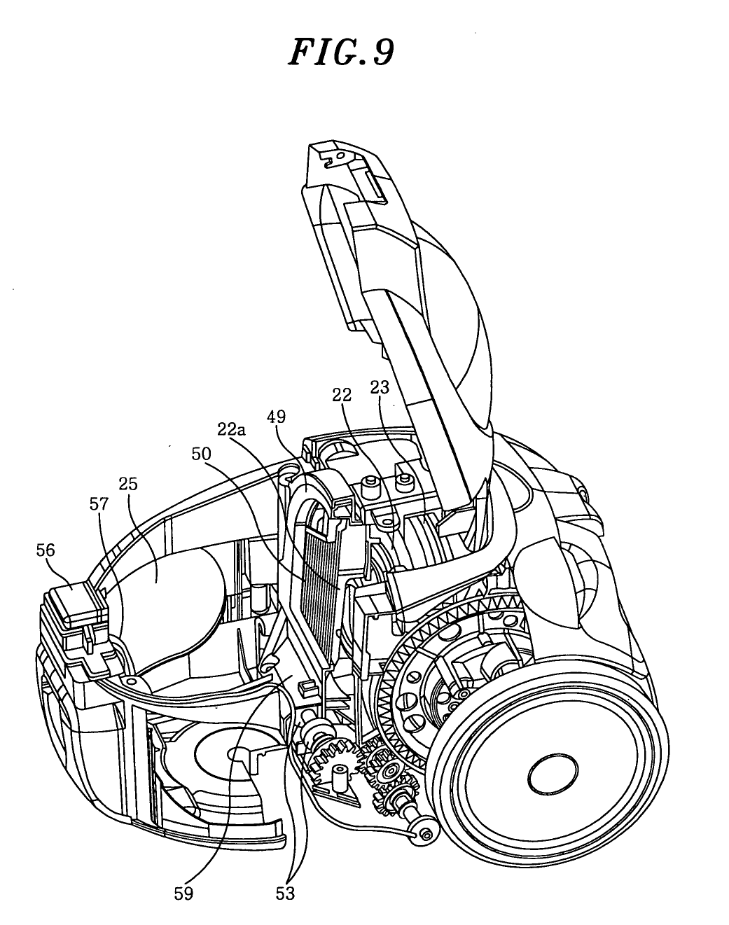

FIG. 9 shows a perspective view of main body 21 after removing dust box unit 24 therefrom. Installed at rear portion of suction air inlet 30 is packing 57 and provided at lower rear portion of dust box receiving portion 25 are dust rollers 53 (rotating body for removing dust particles). Dust rollers 53 rotate in combination with the extraction of power cord 52 to shake dust removing plate (not shown). Reference numeral 59 represents a cover for covering dust rollers 53. If undesired matters such as hairs are attached on dust rollers 53, they can be easily removed after opening cover 59.

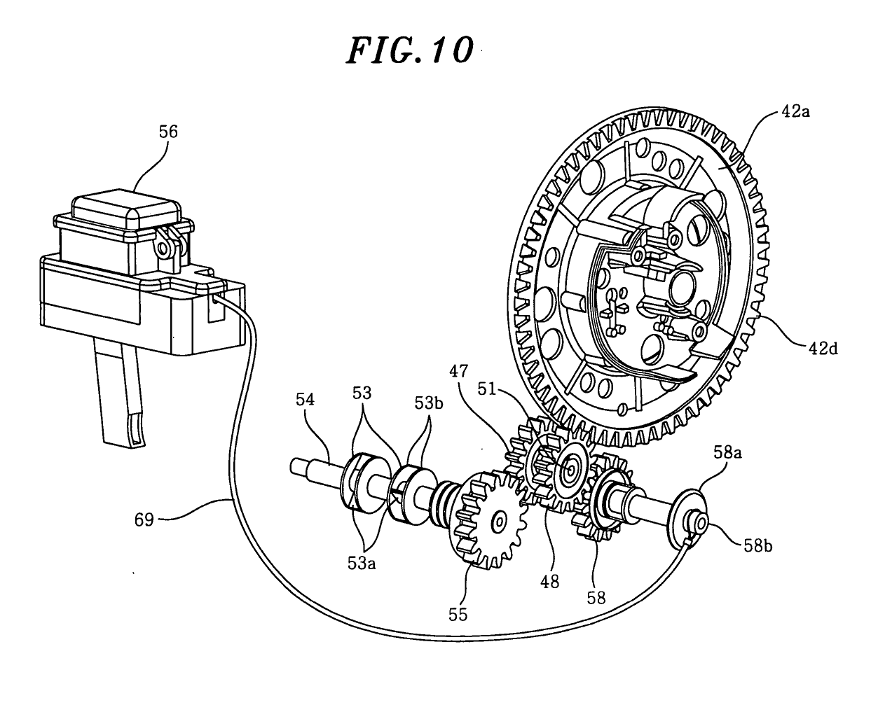

FIG. 10 illustrates a view setting forth of the mechanism of transmitting rotational force to dust rollers 53 and to deodorant accommodating portion 56 when power cord 52 is extracted. One of flanges 42a of cord reel 42 has at its periphery gear tooth 42d in annular shape. Dust rollers 53 are fitted around shaft 54 having at its one end gear 55. Further, gears 47 and 48 are fitted around shaft 51 parallel to each other to rotate simultaneously.

Gears 47 and 48 are positioned between gear tooth 42d and gear 55. In the above mentioned configuration, when flanges 42a of cord reel 43 rotate in response to the extraction of power cord 42, the rotational force thereof is transmitted to dust rollers 53 via gear tooth 42d, gears 48, 47, 55 and shaft 54 in that order, thereby rotating dust rollers 53.

Each of dust rollers 53 has flanges 53b interposing dust pieces 53a therebetween.

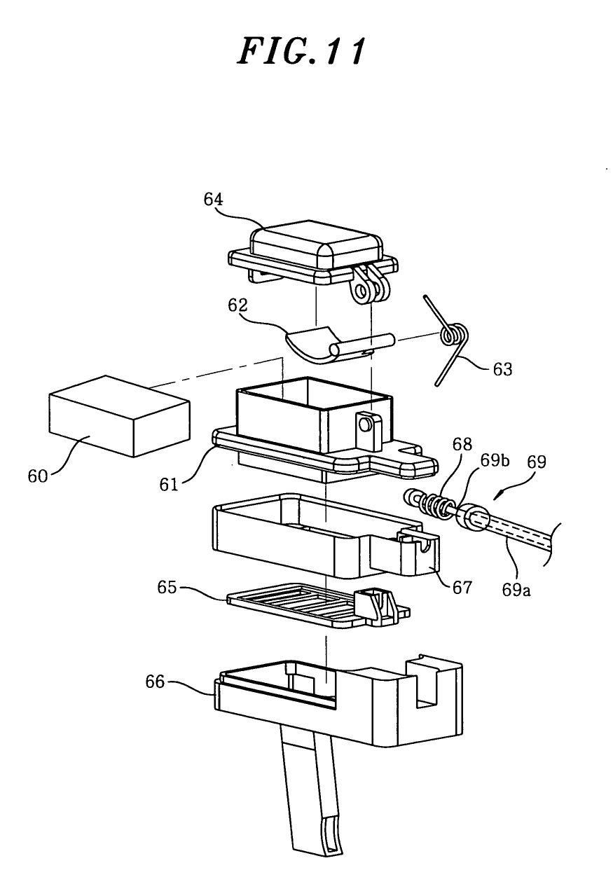

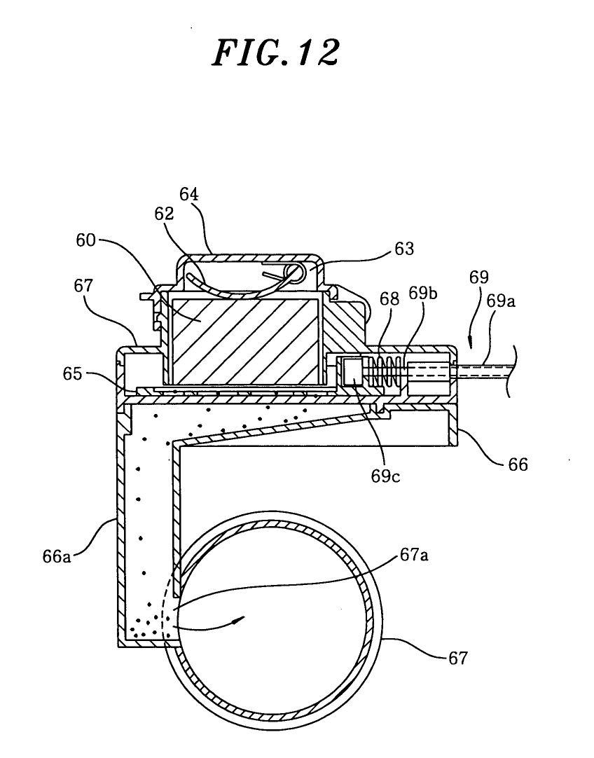

The configuration of deodorant accommodating portion 56 will now be described with reference to FIGS. 11 and 12.

Reference numeral 60 represents a substantially rectangular deodorant wherein deodorant 60 is formed by hardening powered active carbon having an average diameter of 20 μm in the form of substantially rectangular shape by using a colloidal silica binder.

Deodorant 60 is accommodated in recipient box 61 and valve 62 for pressing deodorant 60 is installed above deodorant 60, valve 62 being pivotally installed at lid 64 via spring 63. In addition, lid 64 is axially supported in recipient box 61 to be openable.

Cutter 65, sheath 66, sheath frame 67 and wire part 69 are installed below recipient box 61 wherein cutter 65 is provided with openings for granulating deodorant 60 and wire part 69 is pressed by spring 68 to allow sheath frame 67 and cutter 65 to reciprocate. Wire part 69 has coat 69a and wire 69b slidably inserted into coat 69a. One end of wire 69b is provided with convex portion 69c for loosely fixing cutter 65 and the other end thereof opposite to one end with convex portion is connected to flange 58a coaxially rotated with gear 58 and engagement portion 58b with an eccentric axis center. Sheath 66 is provided at its lower portion with communication path 66a communicating with opening 67a provided at lateral side of packing 57.

Operation of the above mentioned configuration will now be described.

When flanges 42a rotate in response to the extraction of power cord 52, gear tooth 42, gear 48, 58 and flange 58a are rotated by its rotational force. At this time, since engagement portion 58b has an eccentric axis center, wire 69b engaged therewith reciprocates convex portion 69c.

Subsequently, when cutter 65 fixed by convex portion 69c is drawn by wire 69b, spring 68 becomes compressed. When compressed spring 68 returns its initial length, cutter 65 returns its original position as well. By repeatedly doing the above operation, the reciprocation of cutter 35 is repeatedly carried out. Simultaneously, deodorant 60 is cut off and passes through openings of cutter 65, falling down into communication path 66a via sheath 66. When main body 21 is operated, granulated deodorant 60 in communication path 66a is drawn in via opening 67a provided at the lateral side of packing 57 to be mixed with dust particle in dust box unit 24.

Even when there is no cutter 65, deodorant 60 can be broken down and granulated by an impact. At this time, in case the gap between deodorant 60 and recipient 61 is too big, deodorant 60 experiences great movement due to inertia and can be broken down into oversized blocks. Therefore, it is important that the gap is adjusted properly.

The effect of the present invention achieved by the vacuum cleaner with the above configuration will now be described.

The room was cleaned for 15 minutes by the vacuum cleaner in accordance with the present invention. As a comparative experiment, the room was cleaned for 15 minutes by a vacuum cleaner without a deodorant (hereinafter referred to as comparative vacuum cleaner). While closely observing the presence of offensive odor in the exhaust air, the experiments were carried out for 10 days. As a result, exhaust air from the comparative vacuum cleaner began having offensive odor on the fifth day after the experiments; however, the exhaust air from the vacuum cleaner of the present invention had very little smell for 10 days.

When the amounts of dust particles accumulated in the respective vacuum cleaners for 10 days were measured, they were both about 30 g. In case of the inventive vacuum cleaner, the amount of deodorant 60 provided in the inventive vacuum cleaner was reduced by 1.2 g. In other words, both of the vacuum cleaners suction an average of 3 g of dusts a day and the inventive vacuum cleaner uses 0.12 g of deodorant a day.

When the dust particles collected in the inventive vacuum cleaner were observed, deodorant particles were clumped to and disorderly mixed with the dust particles in power form. This is because the deodorant is supplied so that it contacts with and blends with sucked dust particles.

Subsequently, after leaving idle for a day the inventive and the comparative vacuum cleaners each accommodating therein about 30 g of dust particles, they are respectively operated in sealed chambers for 30 seconds. Thereafter, a smell test for offensive odor from exhaust air is performed by six persons in each of sealed chambers. As a result, an average odor intensity level in accordance with the inventive vacuum cleaner was about 1.8 while that according to the comparative one was about 3.0. Therefore, it is understood that the odor reducing efficiency be increased considerably in the present invention.

Considering that 2 is the threshold value of the odor intensity for human perception as an odor, a person will merely perceive a smell at 1.8 without identifying it as an odor. Therefore, it is understood that the inventive deodorization can reduce the offensive odor with superior efficiency.

In the present invention, the offensive odor can be efficiently removed regardless of the cleaning operation of the vacuum cleaner or not by directly loading deodorant into the dust particle sucked in the vacuum cleaner. Therefore, the offensive odor in the dirt correction chamber is reduced and even though the offensive odor level is relatively high, for instance when the cleaning operation of the vacuum cleaner is initiated, the offensive odor of the exhaust air can be decreased. Further, since deodorant is loaded by using the shaving process, dust particle apparatus can be configured simply to facilitate the apparatus miniaturization. Moreover, although the above discussions refer to a situation where the active carbon is used as deodorant, silica, alumina, zeolite or the like is used as deodorant to reduce complex odor, e.g., offensive odor caused by the dust particle of the vacuum cleaner.

In the present invention, since the deodorization efficiency is greatly improved, offensive odors can be minimized in bagless vacuum cleaners as well as ones that use porous bags.

While the invention has been shown and described with respect to the preferred embodiments, it will be understood by those skilled in the art that various changes and modifications may be made without departing from the spirit and scope of the invention as defined in the following claims.

What is claimed is:

1. A method for deodorizing dust particles by mixing the dust particles with a deodorant in the form of granules or powders.

2. A method for deodorizing dust particles, comprising the steps of:
forming a solid deodorant into granules or powders; and
mixing the dust particles with the deodorant in the form of granules or powders.

3. An apparatus for deodorizing dust particles, comprising:
a housing chamber;
a powdering/granulating unit installed in the housing chamber;
a deodorant disposed to be movable relative to the powdering/granulating unit; and
a driving unit for driving the powdering/granulating unit or the deodorant,
wherein the deodorant is formed into granules or powders by a relative movement of the deodorant and the powdering/granulating unit, and thus such deodorant in granule or powder form is mixed with the dust particles.

4. An apparatus for deodorizing dust particles, comprising:
a dust chamber for colleting dust particles therein;
a deodorant to be formed into granules or powders; and
a mixing unit for mixing the dust particles with the deodorant in the form of granules or powders.

5. A vacuum cleaner comprising:
an electric blower for suctioning dust particles;
a dust chamber for collecting the dust particles therein; and
a deodorizing apparatus for supplying a deodorant in the form of granules or powders into the dust chamber or at an upstream location of the dust chamber,
wherein the deodorant in the form of granules or powders is mixed with the dust particles by a suctioning force of the electric blower.

6. The vacuum cleaner of claim 5, further comprising a housing chamber is installed in and made to communicate with the dust chamber or a dust particle passageway upstream of the dust chamber, wherein the deodorant is loaded in the housing chamber to supply the deodorant in the form of granules or powders into the dust chamber.

7. The vacuum cleaner of claim 5, further comprising a powdering/granulating unit for forming a solid deodorant into granules or powders, wherein the solid deodorant is formed into granules or powders by the powdering/granulating unit.

8. The vacuum cleaner of claim 6, further comprising a powdering/granulating unit for forming a solid deodorant into granules or powders, wherein the solid deodorant is formed into granules or powders by the powdering/granulating unit.

9. The vacuum cleaner of claim 7, wherein the deodorant and the powdering/granulating unit are disposed to be movable relative to each other so that at least one of the deodorant and the powdering/granulating unit is moved by an drawing and moving action of a main body of the vacuum cleaner, and the deodorant is formed into granules or powders by the movement of said at least one of the deodorant and the powdering/granulating unit.

10. The vacuum cleaner of claim 8, wherein the deodorant and the powdering/granulating unit are disposed to be movable relative to each other so that at least one of the deodorant and the powdering/granulating unit is moved by an drawing and moving action of a main body of the vacuum cleaner, and the deodorant is formed into granules or powders by the movement of said at least one of the deodorant and the powdering/granulating unit.

11. The vacuum cleaner of claim 7, wherein the deodorant and the powdering/granulating unit are disposed to be movable relative to each other, and at least one of the deodorant and the powdering/granulating unit is driven manually so that the deodorant can be formed into granules or powders at any time a user wishes.

12. The vacuum cleaner of claim 8, wherein the deodorant and the powdering/granulating unit are disposed to be movable relative to each other, and at least one of the deodorant and the powdering/granulating unit is driven manually so that the deodorant can be formed into granules or powders at any time a user wishes.

13. The vacuum cleaner of claim 7, wherein the deodorant and the powdering/granulating unit are disposed to be movable relative to each other, and further comprising an electric driving unit for driving at least one of the deodorant and the powdering/granulating unit and a controller for controlling the electric driving unit to be operated constantly, periodically or intermittently to form the deodorant into granules or powders.

14. The vacuum cleaner of claim 8, wherein the deodorant and the powdering/granulating unit are disposed to be movable relative to each other, and further comprising an electric driving unit for driving at least one of the deodorant and the powdering/granulating unit and a controller for controlling the electric driving unit to be operated constantly, periodically or intermittently to form the deodorant into granules or powders.

15. The vacuum cleaner of claim 7, wherein the deodorant and the powdering/granulating unit are disposed to be movable relative to each other, and further comprising a cord reel for driving at least one of the deodorant and the powdering/deodorizing unit so that the deodorant is formed into granules or powders when a cord is extracted or retracted.

16. The vacuum cleaner of claim 8, wherein the deodorant and the powdering/granulating unit are disposed to be movable relative to each other, and further comprising a cord reel for driving at least one of the deodorant and the powdering/deodorizing unit so that the deodorant is formed into granules or powders when a cord is extracted or retracted.

17. The vacuum cleaner of claim 6, wherein the housing chamber is installed in the dust chamber or at a location above the dust particle passageway upstream of the dust chamber.

18. The vacuum cleaner of claim 6, wherein the housing chamber is provided with an opening for replacing the deodorant with a new one.

19. The vacuum cleaner of claim 5, further comprising a deodorant amount indicator for indicating a residual amount of the deodorant.

20. The vacuum cleaner of claim 5, further comprising at least one of an antibacterial agent and an aromatic compound as well as the deodorant, wherein the deodorant and said at least one of the antibacterial agent and the aromatic compound are formed into granules or powders to be mixed with the dust particles.
